(19) United States
(12) Patent Application Publication
(10) Pub. No.: US 2009/0192595 A1
(43) Pub. Date: Jul. 30, 2009

(54) MEDICAL IMPLANT

(75) Inventors: Hiroaki Nagura, Ashigarakami-gun (JP); Yoshihito Kawamura, Kumamoto-shi (JP); Michiaki Yamasaki, Kumamoto-shi (JP)

Correspondence Address: BUCHANAN, INGERSOLL & ROONEY PC, POST OFFICE BOX 1404, ALEXANDRIA, VA, 22313-1404, US

(73) Assignee: TERUMO KABUSHIKI KAISHA, Shibuya-ku (JP)

(21) Appl. No.: 12/362,258

(22) Filed: Jan. 29, 2009

(30) Foreign Application Priority Data
Jan. 30, 2008 (JP) 2008-019172

(51) Int. Cl. A61F 2/06

(52) U.S. Cl. 623/1.46; 623/1.15

(57) ABSTRACT

Disclosed herein is a medical implant including an implant body of which at least a part is comprised of a biodegradable metal, wherein the part comprised of the biodegradable metal has a crystal grain diameter of not more than 10 μm.

### BACKGROUND OF THE INVENTION

1. Field of the Invention

The present invention relates to a medical implant for use in therapy of a diseased portion of the living body of a human being or other animal.

2. Description of the Related Art

The medical implant relating to the present invention includes a variety of implants such as stent, balloon, cannula, catheter, artificial blood vessel, stent graft, etc. The following description will be made by taking a stent as an example of the medical implant.

A stent is a medical implant which is implanted in a lumen such as a blood vessel, a lymph vessel, a bile duct, a ureter, etc. so as to retain an appropriate lumen diameter and to secure crossability of the lumen.

The material constituting the stent is required to have both of a high strength and a high ductility, which are contradictory properties. If the strength is low, a radial force (strength in the radial direction) required of a stent cannot be obtained. If the ductility is low, on the other hand, the stent would recoil (would contract in diameter in the radial direction after once expanded in the diameter) after implanting in a target site and expanded, so that the intended function of the stent would be spoiled.

In addition, it is preferable that the stent is comprised of a material which will be decomposed in a living organism, namely, a biodegradable material. A stent comprised of a biodegradable material has the advantageous effects of (a) meeting the conceptual request for removing an artificial member from the living organism upon completion of its function, (b) avoiding a chronic inflammatory reaction arising from long-term implanting of a foreign matter (removing mechanical stress), and (c) facilitating retreatment of the lesion portion (re-implanting of the stent); further, for example in the case where the stent is implanted in a blood vessel, it has the advantageous effect of (d) making it possible to cope with variations in the blood vessel (serpentining, vascular sclerosis, ectasis) due to aging, and thereby to suit revascularization.

Furthermore, it is preferable that the rate of decomposition (degradation) of the stent in the living organism can be controlled as desired. For this purpose, the material (composition) of the stent has to be selected.

Thus, a stent is required to be comprised of a biodegradable material which is not limited in composition and which has both a high strength and a high ductility.

As an example of the stent comprised of a biodegradable material, the Igaki-Tamai Stent may be mentioned (refer to, for example, Non-patent Document 1). In this stent, polylactic acid, which is a biodegradable polymer, is used as the material, so that the stent is decomposed in the living body and shows the above-mentioned advantageous effects (a) to (d).

However, such a biodegradable stent comprised of a biodegradable polymer as just-mentioned is low in strength of material. In order to obtain a required radial force by compensating for the low material strength, the thickness of a filamentous member of the stent should be set thicker than that of a metallic stent. When the filamentous member of the stent is made thicker, it adversely affects the properties of the stent, such as deliverability to a lesion site and stimulation on the lesion tissue.

In view of this, a stent produced by a biodegradable metal may be contemplated as a stent capable of solving the above-mentioned problems. For example, magnesium (Mg) is a material which is biodegradable and, simultaneously, has a strength higher than those of biodegradable polymers. Therefore, a stent produced by Mg is expected to ensure that the filamentous member thereof can be made thinner.

In addition, though Mg is low in ductility when used alone, it shows an enhanced ductility when alloyed with lithium (Li) (refer to, for example, Non-patent Document 2). Therefore, a stent produced by such an alloy (Mg—Li alloy) is expected to be able to solve the above-mentioned problems.

A stent comprised of a Mg-Li alloy is described, for example, in Patent Document 1. Patent Document 1 describes a medical implant (inclusive of stent) comprising 50 to 98% of magnesium, 0 to 40% of lithium, 0 to 5% of iron, and less than 5% of other metal or rare earth element.

According to this method, however, the material (composition) of the stent or the like is limited to Mg and Li, etc. Therefore, it is impossible to regulate the composition of the material so as to control, as desired, the decomposition (degradation) rate of the stent in a living organism.

Meanwhile, it is known that when a Mg—Zn alloy contains Zr as an additive element, the alloy shows refined crystal grains and improved mechanical properties, as described for example in Non-patent Document 3. It is also known that an Al-containing Mg alloy can be improved in mechanical properties through grain refining by applying a superheating treatment method or a carbon addition method, as described for example in Non-patent Document 4.

[Patent Document 1]

JP-T-2001-511049

[Non-patent Document 1]

Patrick W. Serruys et al., IGAKI-TAMAI (Registered Trademark) STENT, “HANDBOOK of CORONARY STENTS,” 2000

[Non-patent Document 2]

Takeshi Yoshida et al., ‘Mg—Li Alloys,’ “KINZOKU (Metals),” Jul. 1, 2001, Vol. 71, No. 7, pp. 620-627

[Non-patent Document 3]

Shigeharu Kamatsuchi, Hisashi Obara, Akira Kojima, “Advanced Manufacturing Technologies of Magnesium Alloys,” CMC Publishing Co., Ltd., Feb. 25, 2005, 1st Ed., pp. 20-37

[Non-patent Document 4]

“HANDBOOK of MAGNESIUM TECHNOLOGIES,” edited by The Japan Magnesium Association, Callos Publishing Co., Ltd., May 17, 2000, 1st Ed., pp. 163-165

### SUMMARY OF THE INVENTION

As above-mentioned, when a Mg—Zn alloy contains Zr as an additive element, the alloy shows refined crystal grains and improved mechanical properties. Therefore, a medical implant (such as a stent) produced by such an alloy not only has biodegradability in spite of its being other than Mg—Li alloys but also may have necessary strength and ductility.

However, this medical implant has been improved in mechanical properties through natural refining of crystal grains due to the presence of specific elements such as Mg and Zr contained therein. On the contrary, medical implants in which other elements than the specific elements such as Mg and Zr can be used as material and which are produced through intentional refining of crystal grains in the material have not hitherto been investigated.

Here, to the production of a medical implant, it may be contemplated to apply the above-mentioned method of improving mechanical properties of an Al-containing Mg alloy through refining of crystal graining by applying the superheating treatment technique or the carbon addition technique. However, this method is applicable only where an Al-containing Mg alloy is used, and, therefore, the method has limitations in regard of material. The limitation on material makes it difficult to control the decomposition (degradation) rate of the medical implant in a living organism.

In addition, the superheating treatment technique applied here has the problem of high energy cost required for maintaining the molten metal in a superheated state at a high temperature. Thus, it is difficult to put this method to practical use. Further, the carbon addition technique has many problems; for example, C₂Cl₆ or the like to be used as a grain refining agent is designated as environmentally toxic substance and, therefore, cannot be used.

Besides, heretofore, medical implants in which crystal grains in the material forming an implant body are refined to or below a specified grain size so as to secure strength and ductility preferable for medical implant such as stent have not been investigated.

Accordingly, it is an object of the present invention to provide a medical implant which is biodegradable, is not limited as to the material (composition) of an implant body thereof, and has a strength required of a medical implant, which a biodegradable polymer doesn't have, and a ductility for satisfactorily coping with deformations brought about in implanting the medical implant in a target site.

The present inventors have made researches on medical implants for solving the above-mentioned problems. As a result of their researches, the inventors have found out that the above-mentioned problems involved in the related art can be solved by a medical implant in which an implant body is comprised of a biodegradable material and a part, comprised of a biodegradable metal, of the implant body has a crystal grain diameter of not more than a specified value.

More specifically, the present invention resides in the following (1) to (18).

(1) A medical implant including an implant body of which at least a part is comprised of a biodegradable metal, wherein the part comprised of the biodegradable metal has a crystal grain diameter of not more than 10 μm.

(2) The medical implant as described in (1) above, wherein the part comprised of the biodegradable metal has been subjected to a grain refining treatment.

(3) The medical implant as described in (2) above, wherein the grain refining treatment is an ECAE (Equal Channel Angular Extrusion) treatment.

(4) The medical implant as described in any of (1) to (3) above, wherein the implant body is comprised of the biodegradable metal.

(5) The medical implant as described in any of (1) to (4) above, wherein the biodegradable metal contains Mg.

(6) The medical implant as described in any of (1) to (5) above, wherein the biodegradable metal contains at least one element selected from the biocompatible element group consisting of Zr, Y, Ti, Ta, Nd, Nb, Zn, Ca, Al, Li, Sc and Mn and the rare earth element group consisting of La, Ce, Pr, Sm, Eu, Gd, Tb, Dy, Ho, Er, Tm, Yb and Lu.

(7) The medical implant as described in any of (1) to (5) above, wherein the biodegradable metal is Mg.

(8) The medical implant as described in any of (1) to (7) above, including a layer comprised of a composition of a biological physiologically active substance and a biodegradable polymer, at a surface of the implant body.

(9) The medical implant as described in any of (1) to (8) above, including a layer comprised of a biological physiologically active substance and a layer comprised of a biodegradable polymer, at a surface of the implant body.

(10) The medical implant as described in (8) or (9) above, wherein the biodegradable polymer contains a plasticizer.

(11) The medical implant as described in any of (1) to (10) above, which is a tubular body.

(12) The medical implant as described in any of (1) to (11) above, which is a stent.

(13) The medical implant as described in any of (8) to (12) above, wherein the biological physiologically active substance is at least one selected from the group consisting of carcinostatic agents, immunosuppressors, antibiotics, antirheumatics, antithrombotic agents, HMG-CoA (3-hydroxy-3-methylglutaryl coenzyme A) reductase inhibitors, ACE (angiotensin converting enzyme) inhibitors, calcium antagonists, antilipidemic agents, integrins inhibitors, antiallergic agents, antioxidant agents, GPIIbIIIa antagonists, retinoids, flavonoids, carotenoids, lipid improvers, DNA (Deoxyribonucleic acid) synthesis inhibitors, tyrosine kinase inhibitors, antiplatelet agents, anti-inflammatory agents, bio-derived materials, interferons, and NO production promoting substances.

(14) The medical implant as described in any of (8) to (13) above, wherein the biodegradable polymer is at least one selected from the group consisting of polyglycolic acid, polylactic acid, polycaprolactone, polyhydroxybutyric acid, cellulose, polyvaleric acid hydroxybutylate, and polyorthoester, or a copolymer, mixture or composite compound thereof.

(15) The medical implant as described in any of (10) to (14) above, wherein the plasticizer is at least one selected from the group consisting of polyethylene glycol, polyoxyethylene polyoxypropylene glycol, polyoxyethylene sorbitan monooleate, monoglyceride, and acetylated monoglyceride, or a mixture thereof.

(16) A method of producing the medical implant as described in any of (1) to (15) above, wherein the method including a grain refining treatment step of refining at least a part of crystal grains so that the part of the implant body which is comprised of the biodegradable metal has a crystal grain diameter of not more than 10 μm.

(17) The method of producing the medical implant as described in (16) above, wherein the grain refining treatment step is a strong-strain working treatment step.

(18) The method of producing the medical implant as described in (17) above, wherein the strong-strain working treatment step is an ECAE treatment step.

In the medical implant according to the present invention, the implant body is comprised of a biodegradable material. Therefore, the medical implant disappears completely from the inside of a living organism after implanting in the living organism for a required period of time, so that inflammatory reactions are prevented from being caused by long-term implanting of the medical implant. Consequently, the condition where the lesion portion has been cured can be maintained for a long period of time.

In addition, since the material (composition) of the medical implant is not limited, unlike in the related art, the decomposition (degradation) rate of the medical implant in a living organism can be controlled as desired.

Besides, since the crystal grains of the biodegradable metal are refined, the medical implant shows enhanced strength and ductility and has physical properties necessary for a medical implant.

### BRIEF DESCRIPTION OF THE DRAWINGS

FIG. 1 is a side view showing an embodiment of a stent according to the present invention; and

FIG. 2 is an enlarged cross-sectional view taken along line A-A of FIG. 1.

### DETAILED DESCRIPTION OF THE PREFERRED EMBODIMENT

Now, the medical implant according to the present invention will be described below.

The medical implant of the present invention has an implant body comprised of a biodegradable material (inclusive of biodegradable metal), and that part of the implant body which is comprised of a biodegradable metal has a crystal grain diameter of not more than 10 μm.

The medical implant according to the present invention may be one comprised of the implant body and other part (for example, one that has a layer comprised of a composition of a biological physiologically active substance and a biodegradable polymer, at a surface of the implant body, as will be described later) or may be one composed only of the implant body.

In addition, the implant body comprised of a biodegradable material may include a portion comprised of a biodegradable metal and a portion comprised of a biodegradable material other than a biodegradable metal. Or, alternatively, the implant body may be composed only of a biodegradable metal, as will be described later.

First, “the part comprised of a biodegradable metal” (hereinafter referred to also as “biodegradable metal part”) of the implant body will be described.

The biodegradable metal part of the medical implant according to the present invention means a part of the implant body, that is, a part or member comprised of a biodegradable metal selected from the materials which will be described later. For example, the biodegradable metal part means that surface part of the implant body or that one of members constituting the implant body which is comprised of a biodegradable metal.

The position of the biodegradable metal part in the implant body is not particularly limited, and may be any position (portion), at which strength and ductility are required, of the implant body.

It is to be noted here that the implant body may be composed only of a biodegradable metal. That is, instead of a configuration in which only a part of the implant body is comprised of a biodegradable metal, a configuration in which the implant body is wholly comprised of a biodegradable metal may be adopted. This case, also, is regarded as one of embodiments in which the implant body has a biodegradable metal part. Namely, this case is regarded as being within the scope of the present invention. When the implant body is wholly comprised of a biodegradable metal, the physical properties of the implant body and the period required for complete disappearance of the implant body from the inside of a living organism can be controlled, which naturally is preferable.

The biodegradable metal part has a crystal grain diameter of not more than 10 μm.

Here, the crystal grain diameter is a crystal grain diameter measured by a linear intercept method using a photograph of a structure observed under a transmission electron microscope, a scanning electron microscope or an optical microscope.

The crystal grain diameter of the biodegradable metal part is preferably not more than 5 μm, more preferably not more than 1 μm. Such a range ensures that the implant body is further enhanced in strength and ductility, to have sufficient physical properties as a medical implant.

The biodegradable metal part preferably has a crystal grain diameter controlled to or below 10 μm, as a result of a grain refining treatment step of refining at least a part of the crystal grains. This treatment provides the implant body with further enhanced strength and ductility, so that the implant body is excellent in physical properties required of a medical implant.

The grain refining treatment step herein means a treatment for refining the crystal grains in the biodegradable metal part so as to reduce the crystal grain diameter to or below 10 μm, and is not particularly limited. Examples of a preferable grain refining treatment step include strong-strain working treatment step, among which preferred is the ECAE treatment step.

The crystal grain refining by such a strong-strain working treatment step is not limited to the composition of the material just-mentioned, and may be applied to alloys having a composition preferable from the viewpoint of biocompatibility and decomposition (degradation) rate.

The strong-strain working treatment step is a working method for refining crystal grains by repeatedly giving a plastic strain to a metallic material, without changing the shape thereof. Specific examples of the strong-strain working treatment step include a rolling step and an ECAE step. Incidentally, the metallic material which has undergone the strong-strain working may be annealed.

The ECAE (Equal Channel Angular Extrusion) treatment method step comprises giving an extremely large shear strain to the biodegradable metal in a bent die so as to induce dynamic recrystallization during working, thereby refining the crystal grains. Incidentally, heating may be conducted during the working.

The biodegradable metal part which has undergone the ECAE treatment shows a further reduced crystal grain diameter, which naturally is favorable. By this step, the crystal grain diameter can be reduced to or below 5 μm, in some cases to or below 1 μm.

The ECAE treatment step can be favorably applied to the production of the implant body of the medical implant according to the present invention.

Now, the material of the biodegradable metal will be described below.

The biodegradable metal is not particularly limited insofar as it is decomposed (biodegraded) in the living body of a human being or other animal and it permits the crystal grain diameter in the implant body to be controlled to within the above-mentioned range. The biodegradable metal may be Mg, for example. Further, the biodegradable metal may be at least one element selected from the biocompatible element group consisting of Zr, Y, Ti, Ta, Nd, Nb, Zn, Ca, Al, Li, Sc and Mn, and the rare earth element group consisting of La, Ce, Pr, Sm, Eu, Gd, Tb, Dy, Ho, Er, Tm, Yb and Lu.

Among the above-mentioned examples, Mg is preferably contained in the biodegradable metal. Besides, the content of Mg in the biodegradable metal is preferably 50 to 99 mol %, more preferably 90 to 97 mol %.

With Mg contained in the biodegradable metal, the reactivity of the medical implant of the present invention for reaction with the tissues of a living organism is further lowered, and the medical implant will disappear completely from the inside of the living organism after the lapse of the period for which the medical implant should be implanted in the living organism.

Where the biodegradable metal is composed only of Mg, there is obtained a further advantage that formation of thrombosis can be restrained.

Preferably, the biodegradable metal contains at least one element selected from the biocompatible element group consisting of Zr, Y, Ti, Ta, Nd, Nb, Zn, Ca, Al, Li, Sc and Mn, and an arbitrary combination of these elements.

Besides, preferably, the biodegradable metal contains at least one element selected from the rare earth element group consisting of La, Ce, Pr, Sm, Eu, Gd, Tb, Dy, Ho, Er, Tm, Yb and Lu, and an arbitrary combination of these elements.

Where the implant body contains these elements, the physical properties of the medical implant according to the present invention and the period of implanting the medical implant in a living organism can be controlled.

Among the biocompatible element group and the rare earth element group, particularly, at least one element selected from the biocompatible element group consisting of Zr, Y, Nd, Nb, Ca and Li and an arbitrary combination of these elements are preferably contained in the biodegradable metal.

These biocompatible elements makes it possible to control more strictly the physical properties and the implanting period of the medical implant.

Incidentally, the content of the at least one element selected from the biocompatible element group or the rare earth element group based on the biodegradable material is not more than 50 mass %, preferably not more than 30 mass %, more preferably not more than 20 mass %. When the content is more than 50 mass %, the implant body tends to be lowered in strength.

Now, the biodegradable material will be described below.

The biodegradable material in the medical implant according to the present invention is a concept inclusive of the biodegradable metal, and is a material constituting the implant body.

The other part than the biodegradable metal, of the biodegradable material, is not particularly limited insofar as it is a material not adversely affecting the living body of a human being or other animal to which the medical implant of the present invention is implanted.

For example, carbon, hydroxyapatite, polylactic acid, polyethylene glycol, or the like or a mixture of an arbitrary combination of them can be used as the other part (component) than the biodegradable metal.

It should be noted here that if the content of the other component is high, the implant body would be insufficient in strength. Therefore, the content of the other component than the biodegradable metal in the biodegradable material is not more than 50 mass %, preferably not more than 30 mass %, and more preferably not more than 20 mass %.

One example of the composition of the biodegradable material is a composition containing 50 to 98% of magnesium, 0 to 40% of lithium (Li), 0 to 5% of iron, and 0 to 5% of other metal(s) or rare earth element(s) (cerium, lanthanum, neodymium, praseodymium, etc.).

Another example is a composition containing 79 to 97% of magnesium, 2 to 5% of aluminum, 0 to 12% of lithium (Li), and 1 to 4% of rare earth element(s) (cerium, lanthanum, neodymium, praseodymium, etc.).

A further example is a composition containing 85 to 91% of magnesium, 2% of aluminum, 6 to 12% of lithium (Li), and 1% of rare earth element(s) (cerium, lanthanum, neodymium, praseodymium, etc.).

Yet another example is a composition containing 86 to 97% of magnesium, 2 to 4% of aluminum, 0 to 8% of lithium (Li), 1 to 2% of rare earth element(s) (cerium, lanthanum, neodymium, praseodymium, etc.).

A yet further example is a composition containing 8.5 to 9.5% of aluminum, 0.15 to 0.4% of manganese (Mn), 0.45 to 0.9% of zinc, and the balance of magnesium.

Still another example is a composition containing 4.5 to 5.3% of aluminum, 0.28 to 0.5% of manganese (Mn), and the balance of magnesium.

A still further example is a composition containing 55 to 65% of magnesium, 30 to 40% of lithium (Li), and 0 to 5% of other metal(s) and/or rare earth element(s) (cerium, lanthanum, neodymium, praseodymium, etc.).

Particularly, in the case where the implant body is comprised of the Mg-containing biodegradable metal, when all the other elements than Mg are at least one element selected from the above-mentioned biocompatible element group and the above-mentioned rare earth element group, it is possible to obtain a medical implant which can be so controlled as to implant in a living organism for an arbitrary required period while retaining an arbitrary required mechanical strength. Further, the medical implant disappears completely from the inside of the living body after the lapse of the required period, and it is possible to prevent, without re-operation, bad effects from being exerted on the human body or the like due to the presence of the implant in the living body for longer time than required.

Preferably, the medical implant of the present invention as above has a layer comprised of a composition of a biological physiologically active substance and a biodegradable polymer, at a surface of an implant body.

When such a medical implant of the present invention is implanted at a lesion portion in the living organism, the biological physiologically active substance is sustainedly released to promote curing of the lesion portion, which naturally is favorable.

The compositional ratio (mass ratio) between the biological physiologically active substance and the biodegradable polymer in such a composition is set in the range of 1:99 to 99:1, preferably in the range of 30:70 to 70:30. This ensures that the biological physiologically active substance can be contained in as large an amount as possible, while taking into account the physical properties and decomposability (degradability) of the biodegradable polymer.

Besides, preferably, the medical implant of the present invention has a layer comprised of a biological physiologically active substance and a layer comprised of a biodegradable polymer, at a surface of an implant body. This enables stabilization of the biological physiologically active substance and staged release of the biological physiologically active substance into the living body.

Where the medical implant of the present invention is used in a blood vessel and the biological physiologically active substance is appropriately selected, migration and proliferation of the vascular smooth muscle cells can be restrained, so that restenosis can be prevented from being induced by hypertrophy of the inner membrane of the blood vessel.

In addition, since the implant body is comprised of the biodegradable material and the matters present at the surface thereof are the biological physiologically active substance and the biodegradable polymer, the medical implant disappears completely from the inside of the living organism after implanting for a required period of time, so that inflammatory reactions are prevented from being generated due to long-term implanting of the medical implant in the living body. Therefore, the condition where the lesion portion has been cured can be maintained for a long time.

Besides, Mg and the like constituting the implant body are gradually decomposed (degraded) in the living organism, to form hydroxides. Therefore, if the implant body is present alone in the living organism without being accompanied by the biodegradable polymer, the vicinity of the implant body in the living organism is made to be alkaline. However, where polylactic acid or the like is used as the biodegradable polymer, the polylactic acid or the like is gradually decomposed in the living organism to release an acid, so that, eventually, the vicinity of the implant body in the living organism can be brought close to neutrality by the combined the implant body comprised of Mg or the like with the biodegradable polymer comprised of polylactic acid or the like. Therefore, the implant body would not exert bad effects on the living organism. Further, there is no adverse effect exerted on the biological physiologically active substance. Since the biological physiologically active substance may be deteriorated or denatured in an acidic or alkaline atmosphere, it is preferable that neutrality is maintained.

Particularly, in the case where a layer comprised of the biodegradable polymer is provided at a surface of the implant body and a layer comprised of the biological physiologically active substance is provided on the biodegradable polymer layer, the implant body and the biological physiologically active substance are prevented from making direct contact with each other, so that unnecessary chemical reactions and the like are prevented from occurring therebetween. Consequently, the biological physiologically active substance can be prevented from being deteriorated or denatured.

In this manner, the layer comprised of the biological physiologically active substance and the layer comprised of the biodegradable polymer are formed at a surface of the implant body by the method which will be described later. Here, the thickness of the layer comprised of the biological physiologically active substance is in the range of 1 to 100 μm, preferably 1 to 15 μm, and more preferably 3 to 7 μm. The thickness of the layer comprised of the biodegradable polymer is in the range of 0.1 to 100 μm, preferably 1 to 15 μm, and more preferably 3 to 7 μm. The layers having the thickness values in the just-mentioned ranges ensures easy insertion of the medical implant into a blood vessel or the like, and ensures that the biological physiologically active substance in an amount necessary for curing the lesion portion can be mounted while taking into account the physical properties and decomposability (degradability) of the biodegradable polymer.

Incidentally, in the medical implant according to the present invention, a plurality of layers each comprised of the biological physiologically active substance and a plurality of the layers each comprised of the biodegradable polymer may be provided at a surface of the implant body.

The biological physiologically active substance is not particularly limited, and may be selected as desired, insofar as it restrains stenosis and/or occulusion of a vessel system which might occur when the medical implant of the present invention is implanted at a lesion portion. For example, the biological physiologically active substance may be at least one selected from the group consisting of carcinostatic agents, immunosuppressors, antibiotics, antirheumatics, antithrombotic agents, HMG-CoA reductase inhibitors, ACE inhibitors, calcium antagonists, antilipidemic agents, integrins inhibitors, antiallergic agents, antioxidant agents, GPIIbIIIa antagonists, retinoids, flavonoids, carotenoids, lipid improvers, DNA synthesis inhibitors, tyrosine kinase inhibitors, antiplatelet agents, anti-inflammatory agents, bio-derived materials, interferons, and NO production promoting substances, whereby it is possible to cure a lesion portion through controlling the behavior of cells of the lesion tissues, which naturally is favorable.

Preferred examples of the carcinostatic agent include vincristine, vinblastine, vindesine, irinotecan, pirarubicin, paclitaxel, docetaxel, and methotrexate.

Preferred examples of the immunosuppressor include sirolimus, tacrolimus, azathioprine, cyclosporin, cyclophosphamide, mycophenolate mofetil, everolimus, ABT-578, AP23573, CCI-779, gusperimus, and mizoribine.

Preferred examples of the antibiotics include mitomycin, adriamycin, doxorubicin, actinomycin, daunorubicin, idarubicin, pirarubicin, aclarubicin, epirubicin, peplomycin, and zinostatin stimalamer.

Preferred examples of the antieheumatic include methotrexate, sodium thiomalate, penicillamine, and lobenzarit.

Preferred examples of the antithrombotic agent include heparin, aspirin, antithrombin preparation, ticlopidine, and hirudin.

Preferred examples of the HMG-COA reductase inhibitor include cerivastatin, cerivastatin sodium, atorvastatin, rosuvastatin, pitavastatin, fluvastatin, fluvastatin sodium, simvastatin, lovastatin, and pravastatin.

Preferred examples of the ACE inhibitor include quinapril, perindopril erbumine, trandolapril, cilazapril, temocapril, delapril, enalapril maleate, lisinopril, and captopril.

Preferred examples of calcium antagonist include nifedipine, nilvadipine, diltiazem, benidipine, and nisoldipine.

Preferred examples of the antilipemia agent include probucol.

Preferred examples of the integrins inhibitor include AJM300.

Preferred examples of the antiallergic agent include tranilast.

Preferred examples of the antioxidant include □-tocopherol.

Preferred examples of the GPIIbIIIa antagonist include abciximab.

Preferred examples of the retinoid include all-trans-retinoic acid.

Preferred examples of the flavonoid include epigallocatechin, anthocyanin, and proanthocyanidin.

Preferred examples of the carotenoid include □-carotene, and lycopene.

Preferred examples of the lipid improver include eicosapentaenoic acid.

Preferred examples of the DNA synthesis inhibitor include 5-FU.

Preferred examples of the tyrosine kinase inhibitor include genistein, tyrophostin, erbstatin, and staurosporine.

Preferred examples of the antiplatelet agent include ticlopidine, cilostazol, and clopidogrel.

Preferred examples of the anti-inflammatory agent include dexamethasone, and prednisolone.

Preferred examples of the bio derived material EGF (epidermal growth factor), VEGF (vascular endothelial growth factor), HGF (hepatocyte growth factor), PDGF (platelet derived growth factor), and BFGF (basic fibroblast growth factor).

Preferred examples of the interferon include interferon gamma-1a.

Preferred examples of the NO production promoting substance include L-arginine.

Whether the biological physiologically active substance should be composed of only one biological physiologically active substance or should be composed of a combination of two or more different biological physiologically active substances may be appropriately selected depending on the individual case.

The biodegradable polymer is not particularly limited insofar as it is a polymer gradually decomposed (biodegraded) when the medical implant of the present invention is implanted at a lesion portion and is a polymer not adversely affecting the living organism of a human being or other animal. The biodegradable polymer is preferably at least one selected from the group consisting of polyglycolic acid, polylactic acid, polycaprolactone, polyhydroxybutyric acid, cellulose, polyvaleric acid hydroxybutyrate, and polyorthoester, or a copolymer, mixture or composite compound thereof, which are lower in reactivity for reaction with the tissues and of which the decomposition (degradation) in a living organism can be controlled.

In addition, preferably, the biodegradable polymer contains a plasticizer, whereby it is possible to prevent cracking or exfoliation of the layer containing the biodegradable polymer which might otherwise occur when the medical implant is deformed.

The plasticizer is not particularly limited insofar as it does not exert bad effects on the living body of a human being or other animal. Preferably, the plasticizer is at least one selected from the group consisting of polyethylene glycol, polyoxyethylene polyoxypropylene glycol, polyoxyethylene sorbitan monooleate, monoglyceride, and acetylated monoglyceride, or a mixture thereof. Such a plasticizer is low in reactivity for reaction with the tissues, and can control the physical properties of the layer containing the biodegradable polymer.

The plasticizer is preferably in the range of 0.01 to 80 mass %, more preferably 0.1 to 60 mass %, and further preferably 1 to 40 mass %, based on the biodegradable polymer. In case of such an amount ratio, the plasticizer shows good compatibility with the biodegradable polymer, and makes it possible to appropriately improve the physical properties of the biodegradable polymer.

The kind of the medical implant according to the present invention is not particularly limited insofar as it is an implant for curing a diseased portion in a living body of a human being or other animal and it can be produced from the biodegradable material including the biodegradable metal. Specific examples of the medical implant include stent, covered stent, coil, micro-coil, artificial blood vessel, artificial bone, shield, wire knitting, clip, and plug.

In addition, the medical implant has, for example, a function of supporting the lumen in a hollow organ and/or a vessel system (ureter, bile duct, urethra, uterus, or bronchia).

Besides, the medical implant is, for example, a closer member for connection of hollow spaces or as a closer system for a vessel or vessel system.

In addition, the medical implant is, for example, a fixing or supporting device for momentarily fixing a tissue implant or a tissue transplant.

Besides, the medical implant is, for example, an orthopaedic implant (bolt, nail, wire, plate, articulation, etc.).

In addition, the medical implant is, for example, a stent graft, a vascular anastomotic device, a vascular hemostatic device, aneurysm curing device, an implanted type medical device using a stent as a retaining element.

The shape of the medical implant according to the present invention varies depending on the individual purpose thereof, however, preferably, it is tubular in shape. The tubular shape permits the medical implant to be implanted stably in a lumen such as a blood vessel.

The tubular medical implants include substantially hollow cylindrical ones having an inner surface and an outer surface. More specifically, the substantially hollow cylindrical medical implants include those in which a substantially hollow cylindrical body comprised of the biodegradable material is provided with small holes or those in which wires or fibers comprised of the biodegradable material are knitted into a hollow cylindrical shape.

The length and the diameter of the tubular medical implant vary depending on the use thereof. Normally, the length is 5 to 1000 mm, and the diameter (the diameter of the substantially circular section) is 1 to 50 mm.

In addition, the medical implant according to the present invention is preferably a stent. The stent makes it possible to expand a stenosed lumen to secure a sufficient inside cavity. Besides, the stent can be easily delivered in a blood vessel by contracting the diameter thereof and using a balloon catheter or the like, and the stent has low possibility of foreign matter reactions. Where Mg is used to produce the implant body, Mg ions are released to the surroundings of the stent, whereby an antithrombotic property can be easily developed, and the stent can easily disappear in a living body.

The stent herein include coil-shaped stent, net-shaped stent, tubular stent (in which a tubular body made of a metal or the like is provided with a multiplicity of holes), etc.

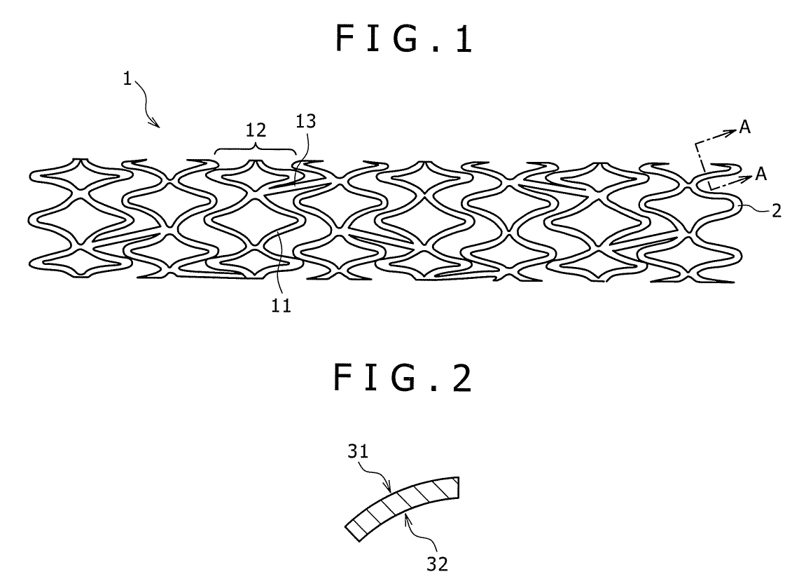

Now, a stent representing a preferred embodiment of the medical implant according to the present invention will be described below, referring to FIGS. 1 and 2. It is to be noted here, however, the stent in the scope of the present invention is not limited to the one described below.

In FIG. 1, the stent 1 is a hollow cylindrical body which is opened at both terminal end portions thereof and which extends in the longitudinal direction between the terminal end portions. A side surface of the hollow cylindrical body has a multiplicity of cutout portions providing communication between an outside surface 31 and an inside surface 32. The hollow cylindrical body can be expanded and contracted in the radial direction through deformation of the cutout portions, and the shape of the hollow cylindrical body is maintained when the stent is implanted in a blood vessel.

In the embodiment shown in FIG. 1, the stent 1 has a basic unit composed of a substantially rhombic element 11 formed from a filamentous member 2 and provided therein with the cutout portion. A plurality of the substantially rhombic elements 11 are continuously arranged and connected in the minor axis direction thereof, to form an annular unit 12. The annular unit 12 is connected to each of the adjacent annular units through a filamentous connecting member 13. Consequently, a plurality of the annular units 12 are arranged in series in the axial direction thereof in the state of being partly connected to one another. The stent 1 thus configured is a hollow cylindrical body which is opened at both terminal end portions thereof and which extends in the longitudinal direction between the terminal end portions. The stent 1 thus has the substantially rhombic cutout portions, so that the stent 1 can be expanded and contacted in the radial direction of the hollow cylindrical body through deformation of the cutout portions.

Incidentally, the above-described stent 1 is merely one embodiment. The medical implant of the present invention widely includes hollow cylindrical body structures which are each comprised of filamentous members to have a sectional shape as shown in FIG. 2 (a sectional shape in which an inside surface 32 forms a shorter arc, while an outside surface 31 forms a slightly longer arc) so as to be opened at both terminal end portions and to extend in the longitudinal direction between the terminal end portions, which has a multiplicity of cutout portions providing communication between the outside surface and the inside surface, and which can be expanded and contracted in the radial direction of the hollow cylindrical body through deformation of the cutout portions.

Now, the method of producing a medical implant according to the present invention will be described below.

The method of producing a medical implant according to the present invention is not particularly limited insofar as it is a method by which an implant body can be produced with a biodegradable metal-made part having a crystal grain diameter of not more than 10 μm.

For example, an ingot of a Mg—Zn alloy with crystal grains refined by addition of Zr thereto is produced, and is polished, to prepare a pipe having a desired size. Then, an opening pattern is adhered to the surface of the pipe, and pipe portions other than the opening pattern are dissolved or melted by an etching technology such as laser etching, chemical etching, etc. to form opening portions. Or, alternately, by a laser beam cutting technology based on pattern information stored in a computer, the pipe can be cut according to a pattern, thereby forming the opening portions.

The method of producing a medical implant of the present invention, preferably, includes a grain refining treatment step of refining at least a part of crystal grains so that the of the implant body which is comprised of the biodegradable metal has a crystal grain diameter of not more than 10 μm.

The method will be described below, taking a tubular stent as an example of the medical implant.

First, the above-mentioned biodegradable metal and, optionally, a biocompatible element, a rare earth element, and an element which does not adversely affect a human body or other animal body are selected, and these materials are melted in an inert gas or vacuum atmosphere. The molten materials are cooled to form an ingot.

The ingot thus obtained is subjected to a grain refining treatment (application of the grain refining treatment step).

The grain refining treatment step is as above-described. A strong-strain working treatment step such as a rolling step and the ECAE treatment step can be preferably applied to the production of the implant body of the medical implant according to the present invention.

The material thus treated is polished, to form a pipe having a desired size. An opening pattern is adhered to the surface of the pipe, and the pipe portions other than the opening pattern are melted or dissolved by an etching technology such as laser etching and chemical etching, to form opening portions. Or, alternately, by a laser beam cutting technology based on pattern information stored in a computer, the pipe can be cut according to the pattern, thereby forming the opening parts.

By such a method, the tubular stent as one example of the medical implant of the present invention can be produced.

Incidentally, in order to form a layer comprised of a composition of the biological physiologically active substance and the biodegradable polymer on the surface of the medical implant of the present invention produced by the above-described method, so as to obtain the medical implant of the present invention in a preferred embodiment, the following operations are carried out.

The biological physiologically active substance and the biodegradable polymer are mixed and dissolved, or are each separately dissolved, in a solvent such as acetone, ethanol, chloroform, tetrahydrofuran, etc. to obtain a solution having a concentration of 0.001 to 20 mass %, preferably 0.01 to 10 mass %, and the solution is applied to the surface of, for example, the stent produced by the above-described method, by a conventional method using a spray, a dispenser or the like, to form a layer on the surface of the stent. Thereafter, the solvent is evaporated off.

The method of using the medical implant of the present invention obtained in the above-mentioned manner is the same as the common method, and is not particularly limited. For example, in the case where a stent as the medical implant of the present invention is used in a blood vessel, for the purpose of expanding a coronary artery narrowed due to arterial sclerosis to thereby improve blood circulation, a method may be adopted in which a balloon catheter is introduced through the femoral artery or brachial artery, the balloon is expanded in a narrowed (stenosed) portion of the blood vessel to expand the blood vessel (circulation reconstructing surgery based on percutaneous coronary intervention), then the balloon is removed, and the stent is inserted to the target portion and is expanded.

Now, the present invention will be described further in detail, based on working examples thereof. It is to be noted that the invention is not limited to the following examples.

### EXAMPLE 1

As a specimen, an AZ31B alloy was prepared.

The specimen was subjected to a solution treatment at 700K for 36000 sec by an electric furnace, then to hot rolling (draft per pass: about 5%; final draft: 50%) at 573K, and to annealing at 473K for 36000 sec.

Here, the specimen after the solution treatment (referred to Specimen 1) and the specimen after the annealing (referred to Specimen 2) were observed microscopically. The microscope was an optical microscope (produced by Leica Inc.), and the magnification was 100 to 1000.

While Specimen 1 had a crystal grain diameter of about 30 to 70 μm, Specimen 2 had a crystal grain diameter of about 3 to 8 μm. Thus, refining of crystal grains was achieved by rolling and annealing.

From Specimens 1 and 2, specimens having a thikness of 0.65 mm, a width of 3 mm and a length of 6 mm were obtained by cutting in parallel to the rolling direction (the thus obtained specimens are referred to respectively as Specimen 10 and Specimen 20), and they were served to tensile tests at room temperature. The results are shown in Table 1 below. It was verified that both strength and ductility are enhanced by refining of crystal grains.

TABLE 1

| | Crystal grain | Tensile | |
|---|---|---|---|
| | diameter | strength | Elongation |
| | (μm) | (MPa) | (%) |
| Specimen 10 | 30-70 | 220 | 18 |
| Specimen 20 | 3-8 | 250 | 30 |

### EXAMPLE 2

From Specimen 2, a square rod member having a thickness of 8 mm, a width of 8 mm and a length of 100 mm was obtained by cutting, and was subjected to centerless polishing, to obtain a round rod member having a diameter of 3 mm. A through-hole with a section diameter of 2.4 mm was bored inside the round rod member by lathe machining, to produce a pipe. The pipe was hot drawn at 573K, to obtain a pipe having an outer diameter of 2 mm and an inner diameter of 1.6 mm.

Upon observation of the pipe under the same microscope as Example 1 and in the same conditions as above, the crystal grain diameter was found to be 2 to 3 μm. Thus, a further refining was confirmed. This is considered to be attributable to dynamic recrystallization during the hot drawing.

### EXAMPLE 3

The pipe produced in Example 2 was subjected to laser beam machining, to produce a stent having a diameter of 2 mm and a length of 15 mm. The stent was expanded to a diameter of 3 mm by a balloon catheter. The stent showed no broken portion even upon expanding. It was thus confirmed that a stent suited to practical use can be produced in this manner.

### EXAMPLE 4

A solution prepared by dissolving a biological physiologically active substance and a biodegradable polymer together with a plasticizer in a solvent was sprayed onto a surface (outer surface) of the same stent as that produced in Example 3.

The biological physiologically active substance used here was sirolimus, which is an immunosuppressor, the biodegradable polymer was polylactic acid (weight average molecular weight: 75000), and the plasticizer was acetylated monoglyceride. These materials were dissolved in acetone in a mass ratio of 5:4:1 so as to obtain a solute concentration of 0.5 mass %.

Then, the acetone used as solvent was completely evaporated off by a vacuum dryer, to form a layer of a mass of about 0.6 mg and a mean thickness of 10 μm on the outside surface of the stent body.

The stent was expanded to a diameter of 3 mm by a balloon catheter in the same manner as in Example 3. The stent showed no broken portion even upon expansion, and the layer composed of the biological physiologically active substance and the biodegradable polymer and the plasticizer showed no cracking or exfoliation. Thus it was confirmed that a stent suited to practical use can be produced in this manner.

### EXAMPLE 5

As specimens, an alloy composed of Mg and Zn and Y was prepared. As for the composition of the alloy, Mg:Zn:Y =96:2:2 (molar ratio).

The specimen was subjected to ordinary extrusion, and further to an ECAE treatment. The conditions of the ordinary extrusion were an extrusion temperature of 350° C. and an extrusion ratio of 10:1. The machining conditions of the ECAE treatment step were an extrusion temperature of 450° C. and the number of times of extrusion of 8. While the specimen before the ordinary extrusion step (referred to as Specimen 3) had a crystal grain diameter of about 30 to 70 μm, the specimen after the ECAE treatment (referred to as Specimen 4) had a crystal grain diameter of about 0.5 to 5 μm. Thus, refining of crystal grains was achieved by the ECAE treatment step.

From Specimens 3 and 4, specimens having a parallel portion length of 15 mm and a diameter of 2.5 mm were obtained by cutting in parallel to the extrusion direction (the thus obtained specimens are referred to respectively as Specimen 30 and Specimen 40), and were served to tensile tests at room temperature. The results are shown in Table 2 below. It was verified that both strength and ductility are enhanced by refining of crystal grains.

TABLE 2

| | Crystal grain | Tensile | Yield | |
|---|---|---|---|---|
| | diameter | strength | strength | Elongation |
| | (μm) | (MPa) | (MPa) | (%) |
| Specimen 30 | 30-70 | 127 | 189 | 3.7 |
| Specimen 40 | 0.5-5 | 350 | 253 | 20.4 |

### EXAMPLE 6

Specimen 4 was worked in the same manner as in Example 2, to obtain a pipe having an outer diameter of 2 mm and an inner diameter of 1.6 mm.

The pipe was subjected to laser beam machining, to prepare a stent having a diameter of 2 mm and a length of 15 mm. The stent was expanded to a diameter of 3 mm by a balloon catheter. The stent showed no broken portion even upon expansion. It was thus confirmed that the stent is suited to practical use.

### COMPARATIVE EXAMPLE

From Specimen 1, a square rod member having a thickness of 8 mm, a width of 8 mm and a length of 100 mm was obtained by cutting, and was subjected to centerless polishing, to obtain a round rod member having a diameter of 2 mm. A through-hole with a section diameter of 1.6 mm was bored inside the round rod member by lathe machining, to produce a pipe. Then, a stent was produced from the pipe in the same conditions as in Example 3.

When the stent was expanded to a diameter of 3 mm by a balloon catheter, the stent was broken at several locations, which showed that it is difficult to put the stent to practical use.

The present invention may be practiced or embodied in still other ways without departing from the spirit or essential character thereof. The preferred embodiments described herein are therefore illustrative and not restrictive, the scope of the invention being indicated by the appended claims and all variations which come within the meaning of the claims are intended to be embraced therein.

What is claimed is:

1. A medical implant comprising an implant body of which at least a part is comprised of a biodegradable metal,
wherein the part comprised of said biodegradable metal has a crystal grain diameter of not more than 10 μm.

2. The medical implant as set forth in claim 1, wherein the part comprised of said biodegradable metal has been subjected to a grain refining treatment.

3. The medical implant as set forth in claim 2, wherein said grain refining treatment is an equal channel angular extrusion treatment.

4. The medical implant as set forth in claim 1, wherein said implant body is comprised of said biodegradable metal.

5. The medical implant as set forth in claim 1, wherein said biodegradable metal contains Mg.

6. The medical implant as set forth in claim 1, wherein said biodegradable metal contains at least one element selected from the biocompatible element group consisting of Zr, Y, Ti, Ta, Nd, Nb, Zn, Ca, Al, Li, Sc and Mn and the rare earth element group consisting of La, Ce, Pr, Sm, Eu, Gd, Tb, Dy, Ho, Er, Tm, Yb and Lu.

7. The medical implant as set forth in claim 1, wherein said biodegradable metal is Mg.

8. The medical implant as set forth in claim 1, comprising a layer comprised of a composition of a biological physiologically active substance and a biodegradable polymer, at a surface of said implant body.

9. The medical implant as set forth in claim 1, comprising a layer comprised of a biological physiologically active substance and a layer comprised of a biodegradable polymer, at a surface of said implant body.

10. The medical implant as set forth in claim 8, wherein said biodegradable polymer contains a plasticizer.

11. The medical implant as set forth in claim 1, which is a tubular body.

12. The medical implant as set forth in claim 1, which is a stent.

13. The medical implant as set forth in claim 8, wherein said biological physiologically active substance is at least one selected from the group consisting of carcinostatic agents, immunosuppressors, antibiotics, antirheumatics, antithrombotic agents, 3-hydroxy-3-methylglutaryl coenzyme A reductase inhibitors, angiotensin converting enzyme inhibitors, calcium antagonists, antilipidemic agents, integrins inhibitors, antiallergic agents, antioxidant agents, GPIIbIIIa antagonists, retinoids, flavonoids, carotenoids, lipid improvers, deoxyribonucleic acid synthesis inhibitors, tyrosine kinase inhibitors, antiplatelet agents, anti-inflammatory agents, bio-derived materials, interferons, and NO production promoting substances.

14. The medical implant as set forth in claim 8, wherein said biodegradable polymer is at least one selected from the group consisting of polyglycolic acid, polylactic acid, polycaprolactone, polyhydroxybutyric acid, cellulose, polyvaleric acid hydroxybutylate, and polyorthoester, or a copolymer, mixture or composite compound thereof.

15. The medical implant as set forth in claim 10, wherein said plasticizer is at least one selected from the group consisting of polyethylene glycol, polyoxyethylene polyoxypropylene glycol, polyoxyethylene sorbitan monooleate, monoglyceride, and acetylated monoglyceride, or a mixture thereof.

16. A method of producing the medical implant as set forth in claim 1,
wherein said method comprising a grain refining treatment step of refining at least a part of crystal grains so that the part of said implant body which is comprised of said biodegradable metal has a crystal grain diameter of not more than 10 μm.

17. The method of producing the medical implant as set forth in claim 16, wherein said grain refining treatment step is a strong-strain working treatment step.

18. The method of producing the medical implant as set forth in claim 17, wherein said strong-strain working treatment step is an equal channel angular extrusion treatment step.
